(19) United States
(12) Patent Application Publication
(10) Pub. No.: US 2026/0021108 A1
(43) Pub. Date: Jan. 22, 2026

(54) USE OF INHIBITORS OF THE HIPPO SIGNALLING PATHWAY FOR THE TREATMENT OF CHRONIC NEPHROPATHIES

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); CENTRE NATIONAL DE LA RECHERCHE SCIENTIFIQUE, Paris (FR); FONDATION IMAGINE, Paris (FR); UNIVERSITÉ PARIS CITÉ, Paris (FR)

(72) Inventors: Amandine VIAU, Paris (FR); Frank BIENAIME, Paris (FR); Giulia FERRI, Paris (FR); Sophie SAUNIER, Paris (FR)

(21) Appl. No.: 19/111,393

(22) PCT Filed: Oct. 2, 2023
(86) PCT No.: PCT/EP2023/077250

§ 371 Date: Mar. 13, 2025

(30) Foreign Application Priority Data
Oct. 3, 2022 (EP) 22306465.0

(51) Int. Cl. A61K 31/551; A61K 31/437; A61P 13/12; A61P 29/00; A61P 37/04

(52) U.S. Cl. CPC A61K 31/551; A61K 31/437; A61P 13/12; A61P 29/00; A61P 37/04

(57) ABSTRACT

Chronic nephropathies, in particular tubulointertial nephropathy or tubulointertial nephropathy with fibrosis feature represent a real global public health concern. In particular, nephronophthisis (NPH) is an orphan genetic disease affecting the kidney. This recessive affection usually manifests with polyuria followed by a gradual reduction in kidney function related to progressive renal scarring. To date, no treatment is available for this affection. Now the inventors show that inhibition of the Hippo signalling pathway represents a new therapeutic avenue for the treatment of chronic nephropathies such as NPH. In particular, the inventors show that inhibition of MST1/2 or LATS1/2 reduces the NPH pro-inflammatory signature in mIMCD-3 renal cells even in response to uropathogenic bacteria. Thus the present invention relates to use of inhibitors of the Hippo signalling pathway for the treatment of chronic nephropathies.

### FIELD OF THE INVENTION

The present invention is in the field of medicine, in particular nephrology.

### BACKGROUND OF THE INVENTION

Chronic nephropathies are defined by the presence of markers of renal damage (structural or functional) and/or a decrease in estimated glomerular filtration rate (eGFR)<60 ml/min/1.73 m² for more than three months. They constitute a real global public health concern due to the constant increase in the prevalence estimated between 10% and 15%.

In particular, nephronophthisis (NPH) is an orphan genetic disease affecting the kidney. This recessive affection usually manifests with polyuria followed by a gradual reduction in kidney function related to progressive renal scarring. To date, no treatment is available for this affection, which is nonetheless the leading genetic cause of end-stage kidney disease in children (König, Jens, et al. “Phenotypic spectrum of children with nephronophthisis and related ciliopathies.” Clinical Journal of the American Society of Nephrology 12.12 (2017): 1974-1983).

NPH is mostly caused by mutations affecting proteins that localize to primary cilia, solitary antenna-like organelles that protrude from the apical surface of most mammalian epithelial cells. Primary cilia emerged from the extension of tubulin doublets originating from the triplets forming the core of the mother centriole. Protein cargoes enter to and exit from the cilia through the transition zone, a complex protein sorting process taking place at the base of the cilium (Davis, Erica E., Martina Brueckner, and Nicholas Katsanis. “The emerging complexity of the vertebrate cilium: new functional roles for an ancient organelle.” Developmental cell 11.1 (2006): 9-19.). The most frequently mutated genes in NPH encode proteins that localize to the transition zone. Indeed, NPHP1, which mutations account for 25% of NPH cases, as well as NPHP4 and RPGRPI1L/NPHP8 are all core proteins of the transition zone (Sang, Liyun, et al. “Mapping the NPH-JBTS-MKS protein network reveals ciliopathy disease genes and pathways.” Cell 145.4 (2011): 513-528.). Loss of function of NPHP genes does not impede ciliogenesis but perturbs cilia organization and/or signalling. Unfortunately, the dysregulated pathways responsible for kidney degeneration in NPH have not yet been solved, precluding the development of efficient therapies for the children and young adults affected by the disease.

The Hippo signalling pathway is an evolutionarily conserved kinase cascade that plays a fundamental role in several biologic processes such as embryonic development, organ size control, cell proliferation and apoptosis (Meng, Zhipeng, Toshiro Moroishi, and Kun-Liang Guan. “Mechanisms of Hippo pathway regulation.” Genes & development 30.1 (2016): 1-17.). The main function of Hippo kinases is to phosphorylate the transcription co-activator YAP (Yes-associated protein) or its paralog TAZ/WWTR1 (Transcriptional coactivator with PDZ-binding domain). Unphosphorylated YAP and TAZ bind to transcriptional enhanced associate domain (TEAD1-4) transcription factors to regulate the expression of multiple genes in a cell and context specific fashion. Upon their phosphorylation by Hippo kinases, YAP and TAZ are targeted to degradation and/or sequestrated in the cytoplasm, shutting down the transcription of their target genes. In mammals, Hippo signalling consists in four serine/threonine kinases: the two upstream mammalian STE20-like protein kinases 1 and 2 (MST1/2; encoded by STK4 and STK3, respectively) phosphorylate the effector large tumor suppressor kinases 1 and 2 (LATS1/2), which in turn phosphorylate YAP and TAZ causing their exclusion from the nuclear compartment (Varelas, Xaralabos. “The Hippo pathway effectors TAZ and YAP in development, homeostasis and disease.” Development 141.8 (2014): 1614-1626.; Ma, Shenghong, et al. “The Hippo pathway: biology and pathophysiology.” Annual review of biochemistry 88 (2019): 577-604.).

Recently, the impact of both genetic and pharmacologic YAP inhibition on the NPH-like phenotype caused by a bi-allelic mutation of Lkb1 was studied (Ferri, Giulia, et al. “YAP restricts renal inflammation and mitigates kidney damage in nephronothisis related kidney disease.” bioRxiv (2022).). It was concluded that YAP inhibition is not a valid therapeutic strategy in NPH and suggest that LKB1 and YAP are parallel negative regulators of a yet uncharacterized pathway detrimental for kidney health.

### SUMMARY OF THE INVENTION

The present invention is defined by the claims. In particular, the present invention relates the use of inhibitors of the Hippo signalling pathway for the treatment of chronic nephropathies.

### DETAILED DESCRIPTION OF THE INVENTION

The first object of the present invention relates to a method of treating a chronic nephropathy in a patient in need thereof comprising administering to the patient a therapeutically effective amount of an inhibitor of the Hippo signalling pathway.

As used herein, the term “patient” refers to a mammalian to which the present invention may be applied. Typically said mammal is a human, but may concern other mammals such as primates, dogs, cats, pigs, sheep, cows. In particular, the term “patient” refers to a mammalian patient, such as a human, who is confirmed to have a chronic nephropathy or who may be classified as having a probable or suspected case of having a chronic nephropathy. In some embodiments, the patient is a human infant. In some embodiments, the patient is a human child. In some embodiments, the patient is a human adult. In some embodiments, the patient is an elderly human.

As used herein, the term “nephropathy” has its general meaning in the art and refers to a physiological condition wherein damage of the kidney occurs that disrupts its ability to properly regulate solute concentrations in the blood and urine. This can be assessed by a number of methods that commonly include: serum creatinine concentration, urinary protein concentration, urinary protein to creatinine ratio or through the use of tracer compounds such as phthalates.

As used herein, the term “chronic nephropathy” refers to a persistent and lasting nephropathy. Chronic nephropathy is defined by the presence of markers of renal damage (structural or functional) and/or a decrease in estimated glomerular filtration rate (eGFR)<60 ml/min/1.73 m² for more than three months (Levey, Andrew S., et al. “The definition, classification, and prognosis of chronic kidney disease: a KDIGO Controversies Conference report.” Kidney international 80.1 (2011): 17-28).

In particular, the patient suffers from tubulointerstitial nephropathy.

As used herein, the term “tubulointerstitial nephropathy” refers to an inflammation of the area of the kidney known as the renal interstitium, which consists of a collection of cells, extracellular matrix, and fluid surrounding the renal tubules.

In particular, the patient suffers from tubulointerstitial nephropathy with fibrosis feature.

As used herein, the term “tubulointerstitial nephropathy with fibrosis feature” is characterized as a progressive detrimental connective tissue deposition on the kidney parenchyma, appears to be a harmful process leading inevitably to renal function deterioration, independently of the primary renal disease which causes the original kidney injury. Tubulointerstitial nephropathy and in particular with fibrosis features includes but are not limited to nephronophthisis, pyelonephritis, obstructive nephropathy or renal ciliopathy.

As used herein, the term “fibrosis” refers to a pathological wound healing in which connective tissue replaces normal parenchymal tissue to the extent that it goes unchecked, leading to considerable tissue remodelling and the formation of permanent scar tissue. Fibrosis comes from the transformation of fibroblasts into myofibroblasts activated by different mechanisms notably by action of pro-fibrotic and pro-inflammatory cytokines released by renal tubular epithelial cells or immune cells.

In some embodiments, the patient suffers from a renal ciliopathy.

As used herein, the term “renal ciliopathy” refers to genetic renal diseases caused by dysfunctional cellular cilia. Renal ciliopathies are thus a group of disorders characterized by nephronophthisis, cystic kidneys or renal cystic dysplasia whose underlying disease pathogenesis is related to abnormal structure or function of the primary cilia complex (Devlin, Laura A., and John A. Sayer. “Renal ciliopathies.” Current Opinion in Genetics & Development 56 (2019): 49-60). Inherited renal ciliopathies include autosomal dominant polycystic kidney disease (ADPKD) and autosomal recessive diseases such as nephronophthisis and autosomal recessive polycystic kidney disease (ARPKD). X-linked disorders, such as oral-facial-digital syndrome secondary to OFD1 mutations, are also part of the renal ciliopathy spectrum.

In some embodiments, the patient suffers from nephronophthisis.

As used herein, the term “nephronophthisis” or “NPH” has its general meaning in the art and refers to a recessive tubulointerstitial ciliopathy that is characterized by a progressive destruction of the kidneys, leading to end stage renal disease (ESRD). NPH is characterized by inflammation and scarring (fibrosis) that impairs kidney function. These abnormalities lead to increased urine production (polyuria), excessive thirst (polydipsia), general weakness, and extreme tiredness (fatigue). The onset of NPH-driven ESRD ranges from the first months of life (infantile NPH) up to >60 years of age (adult NPH), with >17% with ESRD after 20 years of age. Traditionally, the rare disease portal Orphanet reports an approximately world-wide frequency of 1 in 100,000 (Canada 1/50,000, USA 1/900,000, Finland 1/100,000; France 1/50,000). Disease-causing mutations have been identified in more than 23 NPH-associated genes (e.g., NPHP1-20, IFT140, TRAF3IP1/IFT54), accounting for about 60% of all cases presenting with NPH. Full locus deletion of NPHP1 (NPHP 1 (del)) accounts for more than 20% of NPH cases. Mutations in NPHP I are the most common cause of NPH. In a large cohort of patients with adult-onset ESRD (unselected for etiology), NPH due to NPHP I homozygous full gene deletions (NPHP1 (del)) has a prevalence of one in 200 patients (0.5%) in all adult-onset ESRD (Snoek, R. et al., J. Am. Soc. Nephrol., 29:772-9, 2018). Mutations and/or inactivation of one or more of the genes encoding NPHP module proteins may adversely affect ciliogenesis and/or epithelization, resulting in a severe inflammation that leads to fibrosis and cysts development in NPH patients.

In some embodiments, the patient suffers from pyelonephritis.

As used herein, the term “pyelonephritis” has its general meaning in the art and refers to an inflammation of the kidney, typically due to a bacterial infection. Symptoms most often include fever and flank tenderness. Other symptoms may include nausea, burning with urination, and frequent urination. Complications may include pus around the kidney, sepsis, or kidney failure. It is typically due to a bacterial infection, most commonly Escherichia coli.

In some embodiments, the patient suffers from an obstructive nephropathy.

As used herein, the term “obstructive nephropathy” is also known as “uropathy”, and refers to the syndrome caused by urinary tract obstruction, either functional or anatomic. It includes urinary tract dilatation, impedance and the resulting slowing of urine flow, change in the pressure inside the kidney tubular system and impaired kidney function.

As used herein, the term “treatment” or “treat” refers to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In some embodiments, the inhibitors of the Hippo signalling pathway are particularly suitable for reducing inflammation. More particularly, the inhibitors of the Hippo signalling pathway are suitable for reducing the expression and/or secretion of pro-inflammatory cytokines.

As used herein, the term “Hippo signalling pathway” has its general meaning in the art and refers to a kinase cascade known to control organ size through regulation of proliferation and apoptosis. The main function of the Hippo signalling pathway is to phosphorylate the transcription co-activator YAP (Yes-associated protein) or its paralog TAZ/WWTR1 (Transcriptional coactivator with PDZ-binding domain). Unphosphorylated YAP and TAZ bind to transcriptional enhanced associate domain (TEAD1-4) transcription factors to regulate the expression of multiple genes in a cell and context specific fashion. Upon their phosphorylation induced by the Hippo signalling pathway, YAP and TAZ are targeted to degradation and/or sequestrated in the cytoplasm, shutting down the transcription of their target genes. The core components of this pathway include the two upstream mammalian STE20-like protein kinases 1 and 2 (MST1/2; encoded by STK4 and STK3, respectively), the effector large tumor suppressor kinases 1 and 2 (LATS1/2), and the adaptor molecules Salvador homologue 1 (SAVI) and MOB kinase activator 1 (MOB1). Upon activation, MST1/2 phosphorylate LATS1/2. The latter will phosphorylate YAP.

As used herein, the term “inhibitor of the Hippo signalling pathway” refers to any compound that is currently known in the art or that will be identified in the future, and includes any chemical entity that, upon administration to a patient, results in inhibition the Hippo signalling pathway. Ultimately, the inhibitor of the Hippo signalling pathway leads to the activation of the YAP signalling pathway. Inhibitors of the Hippo signalling pathway include but are not limited to low molecular weight inhibitors, antibodies or antibody fragments, antisense constructs, small inhibitory RNAs (i.e. RNA interference by dsRNA; RNAi), and ribozymes. In some embodiments, the inhibitor is a small organic molecule. In some embodiments, the inhibitors of the Hippo signalling pathway include, for example, MST1 or MST2 inhibitors as well as LATS1 or LATS2 inhibitors.

In some embodiments, the inhibitor of the Hippo signalling pathway is a MST1/2 inhibitor. MST1/2 inhibitors are well known in the art and typically include those described in Fan, Fuqin, et al. “Pharmacological targeting of kinases MST1 and MST2 augments tissue repair and regeneration.” Science translational medicine 8.352 (2016): 352ra108-352ra108, Anand, Ruchi, et al. “Toward the development of a potent and selective organoruthenium mammalian sterile 20 kinase inhibitor.” Journal of medicinal chemistry 52.6 (2009): 1602-1611, and in US20120225857, WO2012121992 patent application that are hereby incorporated by reference. Exemplary Mammalian STE20-like kinase inhibitors include the MST1 inhibitors disclosed in US20120225857, Staurosporine, foretinib, bosutinib, KW-2449, crizotinib, NVP-TAE684, cediranib′, AST-487, erlotinib, R406, lestaurtinib, sunitinib, Ki-20227, neratinib, tozasertib, PP-242, R547, doramapimod, brivanib, midostaurin, pazopanib, dovitinib, PHA-665752, ruboxistaurin, linifanib, SU-14813, CHIR-265, fedratinib, JNJ-28312141, gefitinib, axitinib, GSK-461364A, GDC-0879, motesanib, canertinib, ruxolitinib, pictilisib, BMS-387032, BMS-345541, GSK-1838705A, SGX-523, CI-1040, alvocidib, masitinib, A-674563, TG-100-115, GSK690693, VX-745, MLN-120B, tandutinib, MLN-8054, PI-103, selumetinib, barasertib-hQPA, vatalanib, tofacitinib, enzastaurin, lapatinib, SB203580, afatinib, PD-173955, BI-2536, quizartinib, PLX-4720, AT-7519, an anti-Mammalian STE20-like kinase antibody, and an inhibitory Mammalian STE20-like kinase RNA molecule. In some embodiments, the MST1/2 inhibitor is XMU-MP-1 having the UPAC name: 4-[(6, 10-Dihydro-5, 10-dimethyl-6-oxo-5H-pyrimido[5,4-b]thieno[3,2-e][1,4]diazepin-2-yl) amino]benzenesulfonamide.

In a particular embodiment, the inhibitor of the Hippo signalling pathway is not the vandetanib.

In some embodiments, the inhibitor of the Hippo signalling pathway is a LATS1/2 inhibitor. LAST1/2 inhibitors are well known in the art and typically include those described in Kastan, N., Gnedeva, K., Alisch, T. et al. Small-molecule inhibition of Lats kinases may promote Yap-dependent proliferation in postmitotic mammalian tissues. Nat Commun 12, 3100 (2021), Nathaniel Kastan, et al. Small-molecule inhibition of Lats kinases promotes Yap-dependent proliferation in postmitotic mammalian tissues. bioRxiv 2020.02.11.944157 and in Ceribelli, Michele, et al. “Development of selective LATS1 LATS2 inhibitors for the pharmacologic modulation of the Hippo signaling pathway.” MOLECULAR CANCER RESEARCH. Vol. 18. No. 8. 615 CHESTNUT ST, 17TH FLOOR, PHILADELPHIA, PA 19106-4404 USA: AMER ASSOC CANCER RESEARCH, 2020. In particular, the LATS1 inhibitor is N-(3-benzylthiazol-2(3H)-ylidene)-1H-pyrrolo[2,3-b]pyridine-3-carboxamide (TRULI or Lats-IN-1).

As used herein, the term “therapeutically effective amount” refers to an amount effective, at dosages and for periods of time necessary, to achieve a desired therapeutic result. A therapeutically effective amount of drug may vary according to factors such as the disease state, age, sex, and weight of the individual, and the ability of drug to elicit a desired response in the individual. A therapeutically effective amount is also one in which any toxic or detrimental effects of the antibody or antibody portion are outweighed by the therapeutically beneficial effects. The efficient dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required in order to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above. For example, a therapeutically effective amount for therapeutic use may be measured by its ability to stabilize the progression of disease. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected. An exemplary, non-limiting range for a therapeutically effective amount of drug is about 0.1-100 mg/kg, such as about 0.1-50 mg/kg, for example about 0.1-20 mg/kg, such as about 0.1-10 mg/kg, for instance about 0.5, about such as 0.3, about 1, about 3 mg/kg, about 5 mg/kg or about 8 mg/kg. An exemplary, non-limiting range for a therapeutically effective amount of an antibody of the present invention is 0.02-100 mg/kg, such as about 0.02-30 mg/kg, such as about 0.05-10 mg/kg or 0.1-3 mg/kg, for example about 0.5-2 mg/kg. Administration may e.g. be intravenous, intramuscular, intraperitoneal, or subcutaneous, and for instance administered proximal to the site of the target. Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. In some embodiments, the efficacy of the treatment is monitored during the therapy, e.g. at predefined points in time. As non-limiting examples, treatment according to the present invention may be provided as a daily dosage of the inhibitor of the present invention in an amount of about 0.1-100 mg/kg, such as 0.2, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of days 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least one of weeks 1, 2, 3, 4, 5, 6, 7, 8, 9, 10, 11, 12, 13, 14, 15, 16, 17, 18, 19 or 20 after initiation of treatment, or any combination thereof, using single or divided doses every 24, 12, 8, 6, 4, or 2 hours, or any combination thereof.

Typically the inhibitor of the present invention is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The invention will be further illustrated by the following figures and examples. However, these examples and figures should not be interpreted in any way as limiting the scope of the present invention.

### FIGURES

FIG. 1: Pharmacological inhibition of MST1/2 suppresses NPH-cytokine signature in mIMCD-3 renal cells. (A-M) Quantification of Ctgf (A), Cyr61 (B), Ccl2 (C), Ccl5 (D), Cx3cl1 (E), Cxcl1 (F), Cxcl10 (G), Cxcl16 (H), Cxcl17 (I), Il1rn (J), 1133 (K), 1134 (L) and Lgals9 (M) mRNA abundance in confluent mIMCD-3 cells treated or not with 5 μM XMU-MP-1, a specific MST1/2 inhibitor, for 6 hours (n=3). Bars indicate mean±SD. Unpaired t-test, * P<0.05, ** P<0.01, *** P<0.001. AU: arbitrary unit. (N) Heatmap showing the relative mRNA expression (Z-scores) generated on the previous quantitative PCR results.

FIG. 2: MST1/2 inhibition blunts inflammatory response to uropathogenic bacteria in mIMCD-3 renal cells. (A-K) Quantification of Ccl2 (A), Ccl5 (B), Cx3cl1 (C), Cxcl1 (D), Cxcl10 (E), Cxcl16 (F), Cxcl17 (G), Il1rn (H), 1133 (I), 1134 (J) and Lgals9 (K) mRNA abundance in confluent mIMCD-3 cells treated or not with 5 μM XMU-MP-1 and heat inactivated uropathogenic Escherichia coli (UPEC) for 6 hours (n=7-10). Bars indicate mean±SD. One-way ANOVA followed by Tukey test, * P<0.05, ** P<0.01, *** P<0.001, **** P <0.0001. AU: arbitrary unit. (L) Heatmap showing the relative mRNA expression (Z-scores) generated on 3 experiments of the previous quantitative PCR results.

FIG. 3: Pharmacological inhibition of LATS1/2 suppresses partially NPH-cytokine signature in mIMCD-3 renal cells. (A-M) Quantification of Ctgf (A), Cyr61 (B), Ccl2 (C), Ccl5 (D), Cx3cl1 (E), Cxcl1 (F), Cxcl10 (G), Cxcl16 (H), Cxcl17 (I), Il1rn (J), 1133 (K), 1134 (L) and Lgals9 (M) mRNA abundance in confluent mIMCD-3 cells treated or not with 10 μM Lats-IN-1, a specific LATS1/2 inhibitor, for 6 hours (n=3). Bars indicate mean±SD. Unpaired t-test, * P<0.05, ** P<0.01, *** P<0.001. AU: arbitrary unit. (N) Heatmap showing the relative mRNA expression (Z-scores) generated on the previous quantitative PCR results.

FIG. 4: Hippo pathway inhibition reduces NPH-cytokine signature and inflammatory response to bacteria in MDCK renal cells. (A-J) Quantification of Ctgf (A), Cyr61 (B), Ccl2 (C), Cc15 (D), Cx3cl1 (E), Cxcl10 (F), Cxcl16 (G), Cxcl17 (H), Il1rn (I) and 1134 (J) mRNA abundance in confluent MDCK cells treated or not with 5 μM XMU-MP-1 for 6 hours (n=3-6). Bars indicate mean±SD. Unpaired t-test, ** P<0.01, *** P<0.001, **** P<0.0001. AU: arbitrary unit. (K) Heatmap showing the relative mRNA expression (Z-scores) generated on 3 experiments of the previous quantitative PCR results. (L-M) Quantification of Ccl2 (L) and Cxcl10 (M) mRNA abundance in confluent MDCK cells treated with 5 μM XMU-MP-1 or 10 μM Lats-IN-1 and UPEC for 6 hours (n=5). Bars indicate mean±SD. AU: arbitrary unit.

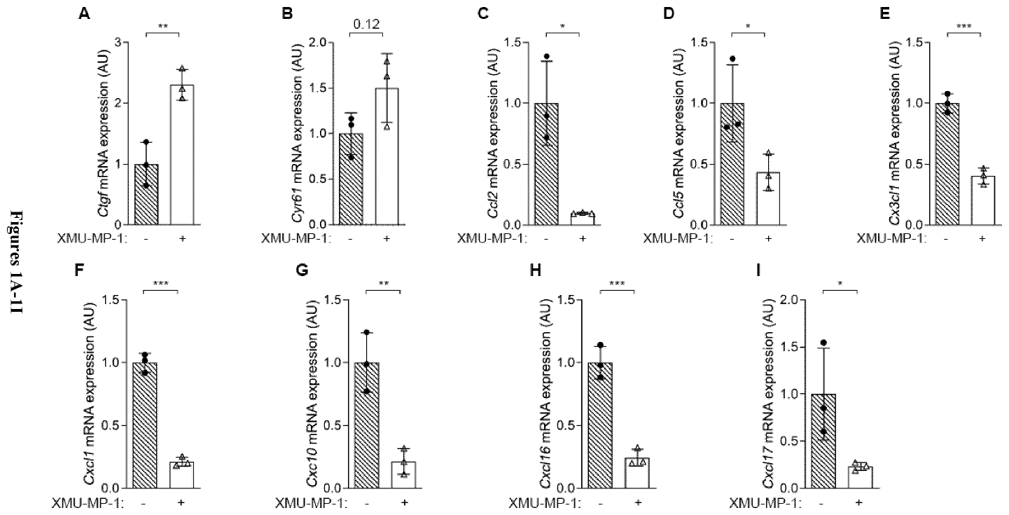

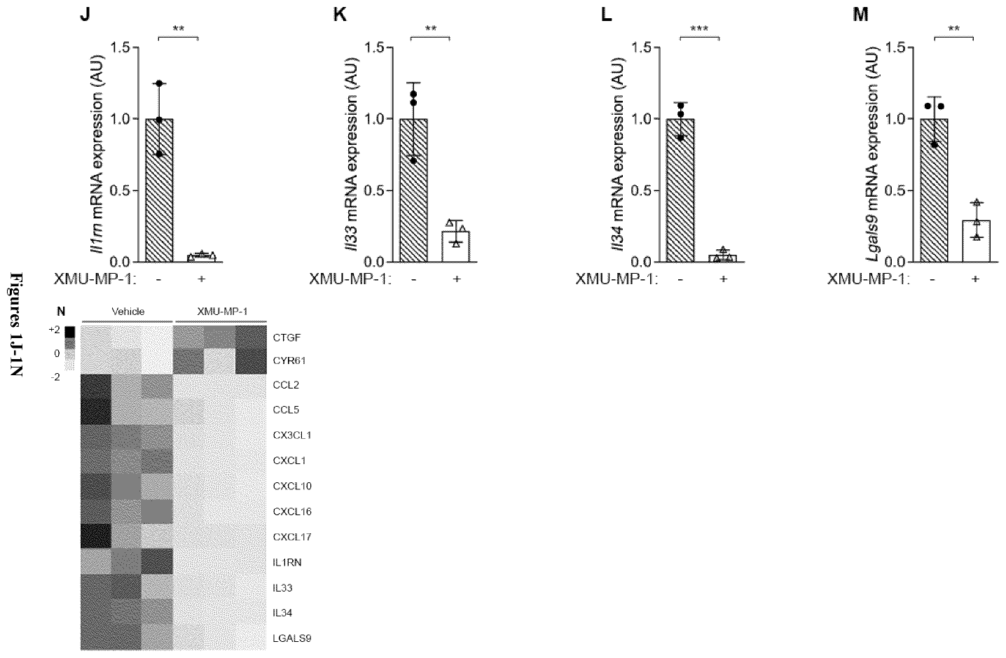

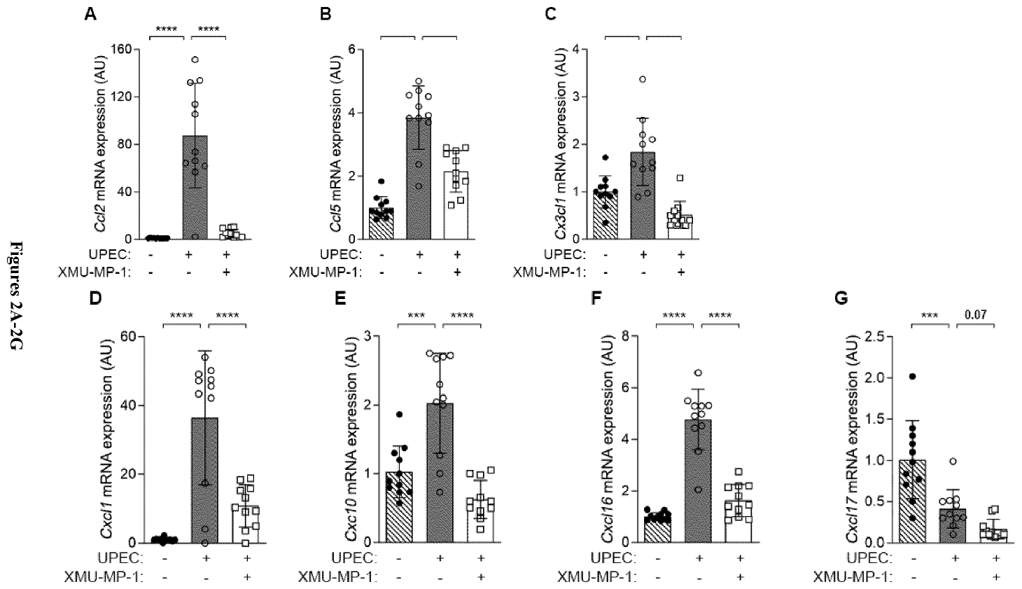

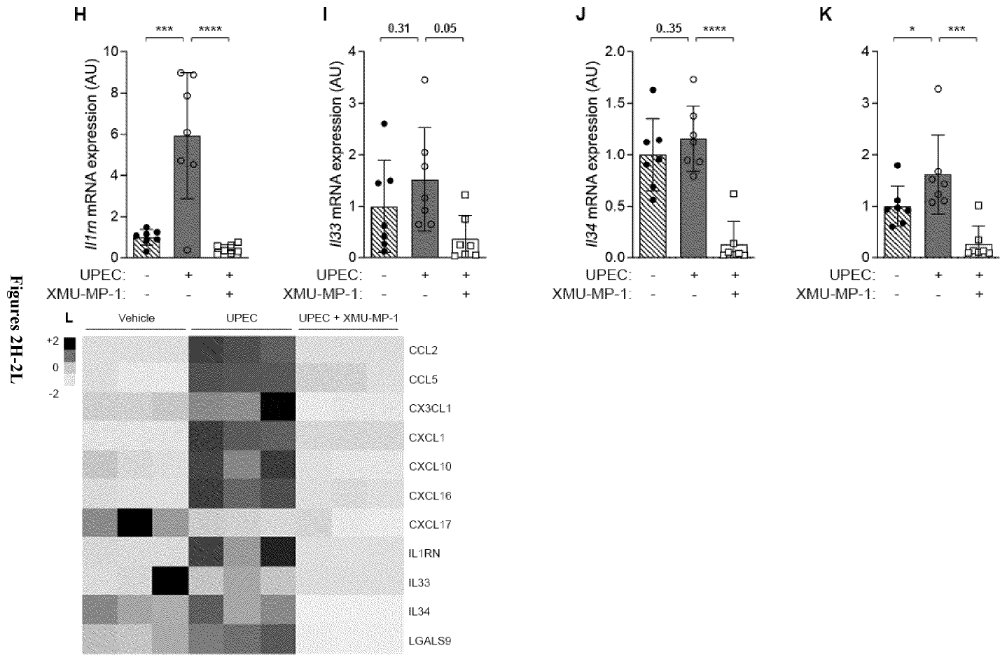

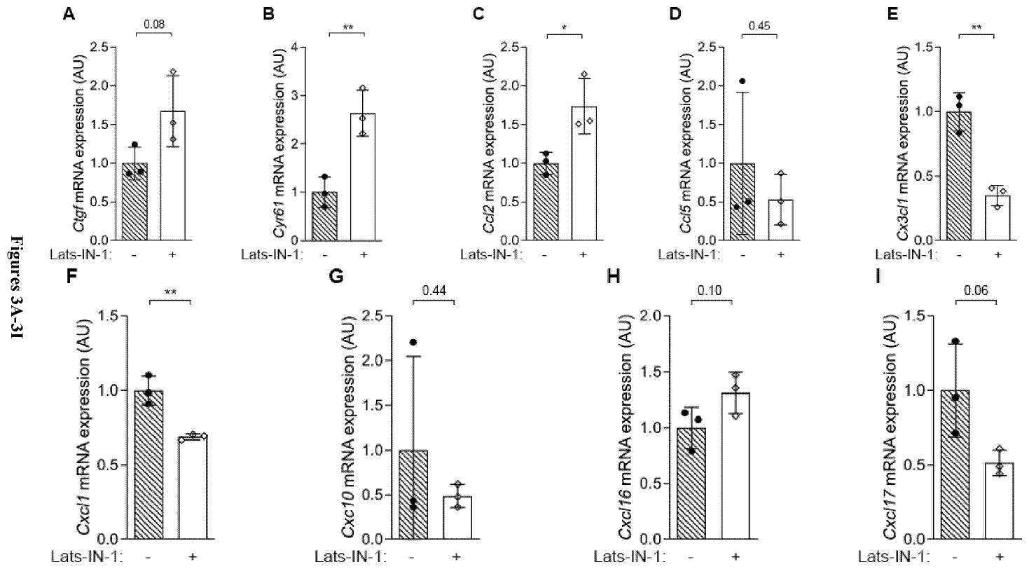

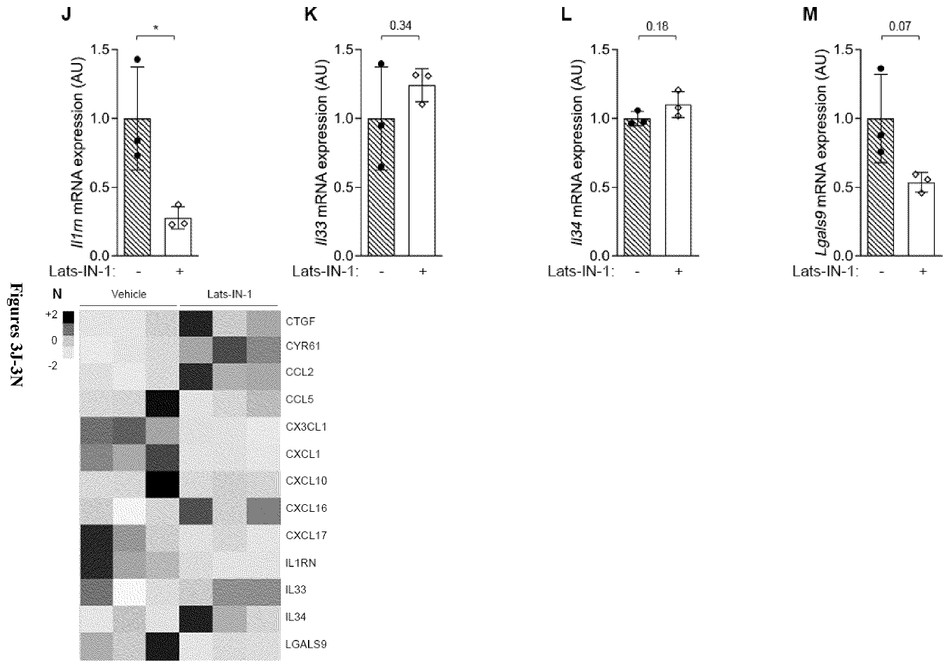

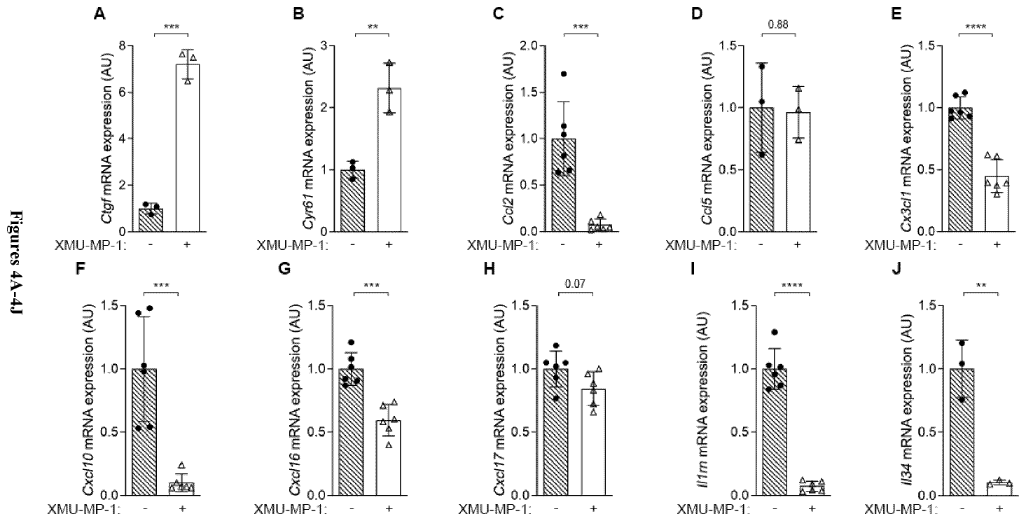

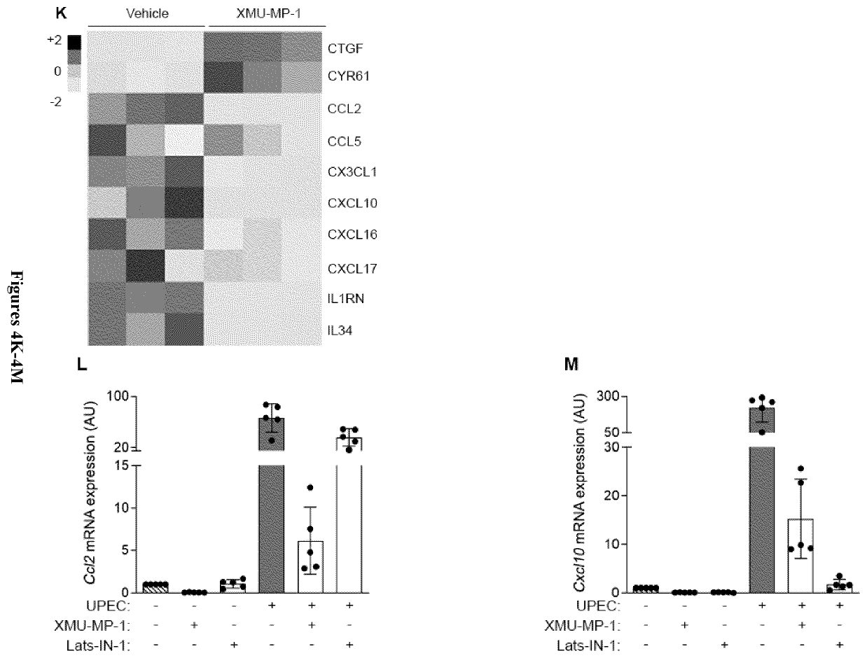

### EXAMPLE

### Methods

### Bacteria Culture

Uropathogen E. coli (UTI89 strain, UPEC) bacteria strains were grown as previously described (Zychlinsky Scharff et al., 2019). Inactivation of bacteria was performed on bacteria resuspended at a concentration of 109/ml in PBS and heated at 60° C. for one hour.

### Cell Culture

Mouse inner medullary collecting duct (mIMCD-3) cells were grown in DMEM/F-12 (1:1, GIBCO, 21331-020) supplemented with 10% FBS, 1% penicillin-streptomycin and 2 mM L-Glutamine (GIBCO, 25030-024). A total of 50,000 cells/cm² were seeded for 3 days on 12 well plate. Confluent mIMCD-3 cells were stimulated with 5 μM XMUMP-1 (Selleckchem, S8334), or 10 μM Lats-IN-1 (MedChemExpress, HY-138489) or 0.04% DMSO. For bacteria experiment, mIMCD-3 cells were starved overnight prior stimulation and then stimulated for 6 hours with 5 μM XMU-MP-1 or 0.04% DMSO with or without 1.108/mL UPEC.

Madin-Darby canine kidney cells (MDCK, kind gift from Prof. Kai Simons, MPI-CBG, Dresden, Germany) were cultured using DMEM (GIBCO, 41966-029) supplemented with 10% FBS (GIBCO, 10270-106) and 1% penicillin-streptomycin (GIBCO, 15140-122). A total of 200,000 cells/cm² were seeded for 10 days on 12 well plate (Corning, 353043). Confluent MDCK cells were stimulated for 6 hours with 5 μM XMU-MP-1 (Selleckchem, S8334) or 0.04% DMSO. For bacteria experiment, MDCK cells were starved overnight prior stimulation and then stimulated for 6 hours with 5 μM XMU-MP-1 or 10 μM Lats-IN-1 (MedChemExpress, HY-138489) with or without 1.108/mL UPEC. All cells were regularly tested for mycoplasma contamination and were mycoplasma-free.

### Quantitative RT-PCR

Total RNAs were obtained from cells using RNeasy Mini Kit (Qiagen) and reverse transcribed using High Capacity cDNA Reverse Transcription Kit (Applied Biosystems) according to the manufacturer's protocol. Quantitative PCR were performed with iTaq™ Universal SYBR® Green Supermix (Bio-Rad) on a CFX384 C1000 Touch (Bio-Rad). Hprt, Sdha, Gapdh and Ppia were used as normalization controls. Each biological replicate was measured in technical duplicates. The primers used for qRT-PCR are listed in Table 1.

### Statistical Analysis

Data are expressed as means+/−standard deviation. Differences between groups were evaluated using unpaired t test when only 2 groups were evaluated or one-way ANOVA followed, when significant (P<0.05), by the Tukey-Kramer test. The statistical analysis was performed using GraphPad Prism V8 software.

TABLE 1
Primer pairs used for qRT-PCR (1/2)

| Gene | | Forward Primer | Reverse Primer |
|---|---|---|---|
| name | Specie | (5′ to 3′) | (5′ to 3′) |
| Ccl2 | Mouse | AGTAGGCTGGAGAGCTACAA | GTATGTCTGGACCCATTCCTTC |
| | | (SEQ ID NO: 1) | (SEQ ID NO: 2) |
| Ccl5 | Mouse | CCAATCTTGCAGTCGTGTTTG | ACCCTCTATCCTAGCTCATCTC |
| | | (SEQ ID NO: 3) | (SEQ ID NO: 4) |
| Ctgf | Mouse | GCTGACCTGGAGGAAAACATTAA | TGACAGGCTTGGCGATTTTAG |
| | | (SEQ ID NO: 5) | (SEQ ID NO: 6) |
| Cx3cl1 | Mouse | GCTTTGCTCATCCGCTATCA | GTCTTGGACCCATTTCTCCTTC |
| | | (SEQ ID NO: 7) | (SEQ ID NO: 8) |
| Cxcl1 | Mouse | CGAAGTCATAGCCACACTCAA | GAGCAGTCTGTCTTCTTTCTCC |
| | | (SEQ ID NO: 9) | (SEQ ID NO: 10) |
| Cxcl10 | Mouse | GGCCATAGGGAAGCTTGAAA | CAGACATCTCTGCTCATCATTCT |
| | | (SEQ ID NO: 11) | (SEQ ID NO: 12) |
| Cxcl16 | Mouse | ATCAGGTTCCAGTTGCAGTC | CATGACCAGTTCCACACTCTT |
| | | (SEQ ID NO: 13) | (SEQ ID NO: 14) |
| Cxcl17 | Mouse | CCTTCCTTCTGTTGCTTCCA | TTCCAAGAGCCACCTCCTA |
| | | (SEQ ID NO: 15) | (SEQ ID NO: 16) |
| Cyr61 | Mouse | GGGTTGGAATGCAATTTCGG | TGGTGTTTACAGTTGGGCTG |
| | | (SEQ ID NO: 17) | (SEQ ID NO: 18) |
| Hprt | Mouse | GTTAAGCAGTACAGCCCCAAA | AGGGCATATCCAACAACAAACTT |
| | | (SEQ ID NO: 19) | (SEQ ID NO: 20) |
| Il1rn | Mouse | TTGTGCCAAGTCTGGAGATG | CTCAGAGCGGATGAAGGTAAAG |
| | | (SEQ ID NO: 21) | (SEQ ID NO: 22) |
| Il33 | Mouse | TGCCTCCCTGAGTACATACA | CTGGTCTTGCTCTTGGTCTTT |
| | | (SEQ ID NO: 23) | (SEQ ID NO: 24) |
| Il34 | Mouse | GATATGGACTCTGACCCAAGATAAG | AGCAATCCTGTAGTTGATGGG |
| | | (SEQ ID NO: 25) | (SEQ ID NO: 26) |
| Lgals9 | Mouse | CAGATGCTACGAGGTTCCATATC | GTGTTTCGGACAACAGCATTC |
| | | (SEQ ID NO: 27) | (SEQ ID NO: 28) |
| Ppia | Mouse | GGCTATAAGGGTTCCTCCTTTC | TTTCTCTCCGTAGATGGACCT |
| | | (SEQ ID NO: 29) | (SEQ ID NO: 30) |
| Sdha | Mouse | AGAACATCAGAACTACGCCTAAACATG | CCATTCCCCTGTCGAATGTCT |
| | | (SEQ ID NO: 31) | (SEQ ID NO: 32) |
| Ccl2 | Dog | CTGCTGCTATACACTCACCAATA | TCAGCACAGATCTCCTTGTTTAG |
| | | (SEQ ID NO: 33) | (SEQ ID NO: 34) |
| Ccl5 | Dog | GCCTCTGCCTCCCCATATG | GGCCGGAAATGTAGGCAAA |
| | | (SEQ ID NO: 35) | (SEQ ID NO: 36) |
| Ctgf | Dog | GCTGACCTGGAAGAGAACATTA | GGTACAACCAGAAAGCTCAAAC |
| | | (SEQ ID NO: 37) | (SEQ ID NO: 38) |
| Cx3cl1 | Dog | GGTCCACTACCAACGAAATCA | CTCCTTTGGATCAGCACAGAA |
| | | (SEQ ID NO: 39) | (SEQ ID NO: 40) |
| Cxcl10 | Dog | TGAAATGATTCCTGCAAGTCCAT | TCTCCCCACTCTTTTTCATTGTG |
| | | (SEQ ID NO: 41) | (SEQ ID NO: 42) |
| Cxcl16 | Dog | CTACGTCAGGTTTCAGCTACG | TCAGCAGAGCCACATTCTTT |
| | | (SEQ ID NO: 43) | (SEQ ID NO: 44) |
| Cxcl17 | Dog | GCAAAGACTGGTTCCTGAGA | CTTCACATTGGCCTTGAAATGA |
| | | (SEQ ID NO: 45) | (SEQ ID NO: 46) |
| Cyr61 | Dog | GTGTACAGCAGCCTGAAGAA | CTCGAACATCCAGCGTAAGTAA |
| | | (SEQ ID NO: 47) | (SEQ ID NO: 48) |
| Gapdh | Dog | CATGTTTGTGATGGGCGTGAACCA | TTTGGCTAGAGGAGCCAAGCAGTT |
| | | (SEQ ID NO: 49) | (SEQ ID NO: 50) |
| Hprt | Dog | AATGTCTTGATTGTTGAGGATAT | ACAAAGTCAGGTTTATAGCC |
| | | (SEQ ID NO: 51) | (SEQ ID NO: 52) |
| Il1rn | Dog | CTGTGTCAAGTCTGGAGATGAG | GCTTGTCTTGATCCTTGTTCTTAC |
| | | (SEQ ID NO: 53) | (SEQ ID NO: 54) |
| Il34 | Dog | CAAAGCCCTCCTGGACAA | TCACAGTCCTGCCAGTTTAG |
| | | (SEQ ID NO: 55) | (SEQ ID NO: 56) |
| Sdha | Dog | TGGGTCCATCCATCGCATA | GAAGTAGGTGCGCCCATAACC |
| | | (SEQ ID NO: 57) | (SEQ ID NO: 58) |

### REFERENCES

Throughout this application, various references describe the state of the art to which this invention pertains. The disclosures of these references are hereby incorporated by reference into the present disclosure.

1. A method of treating a chronic nephropathy in a patient in need thereof comprising administering to the patient a therapeutically effective amount of an inhibitor of the Hippo signaling pathway.

2. The method of claim 1 wherein the inhibitor of the Hippo signaling pathway reduces inflammation.

3. The method of claim 2 wherein the inhibitor of the Hippo signaling pathway reduces the expression and/or secretion of pro-inflammatory cytokines.

4. The method of claim 1 wherein the patient suffers from tubulointerstitial nephropathy.

5. The method of claim 1 wherein the patient suffers from tubulointerstitial nephropathy with fibrosis.

6. The method according to claim 1, wherein the patient suffers from a renal ciliopathy.

7. The method according to claim 1, wherein the patient suffers from nephronophthisis.

8. The method according to claim 1, wherein the patient suffers from pyelonephritis.

9. The method according to claim 1, wherein the patient suffers from the patient suffers from an obstructive nephropathy.

10. The method according to claim 1, wherein the inhibitor of the Hippo signaling pathway is a MST1/2 inhibitor.

11. The method according to claim 1, wherein the inhibitor of the Hippo signaling pathway is a LATS1/2 inhibitor.


SEQUENCE LISTING (58 sequences)

SEQ ID NO: 1 - DNA, 20 bp, synthetic construct
  source 1..20: mol_type = other DNA
agtaggctggagagctacaa

SEQ ID NO: 2 - DNA, 22 bp, synthetic construct
  source 1..22: mol_type = other DNA
gtatgtctggacccattccttc

SEQ ID NO: 3 - DNA, 21 bp, synthetic construct
  source 1..21: mol_type = other DNA
ccaatcttgcagtcgtgtttg

SEQ ID NO: 4 - DNA, 22 bp, synthetic construct
  source 1..22: mol_type = other DNA
accctctatcctagctcatctc

SEQ ID NO: 5 - DNA, 23 bp, synthetic construct
  source 1..23: mol_type = other DNA
gctgacctggaggaaaacattaa

SEQ ID NO: 6 - DNA, 21 bp, synthetic construct
  source 1..21: mol_type = other DNA
tgacaggcttggcgattttag

SEQ ID NO: 7 - DNA, 20 bp, synthetic construct
  source 1..20: mol_type = other DNA
gctttgctcatccgctatca

SEQ ID NO: 8 - DNA, 22 bp, synthetic construct
  source 1..22: mol_type = other DNA
gtcttggacccatttctccttc

SEQ ID NO: 9 - DNA, 21 bp, synthetic construct
  source 1..21: mol_type = other DNA
cgaagtcatagccacactcaa

SEQ ID NO: 10 - DNA, 22 bp, synthetic construct
  source 1..22: mol_type = other DNA
gagcagtctgtcttctttctcc

SEQ ID NO: 11 - DNA, 20 bp, synthetic construct
  source 1..20: mol_type = other DNA
ggccatagggaagcttgaaa

SEQ ID NO: 12 - DNA, 23 bp, synthetic construct
  source 1..23: mol_type = other DNA
cagacatctctgctcatcattct

SEQ ID NO: 13 - DNA, 20 bp, synthetic construct
  source 1..20: mol_type = other DNA
atcaggttccagttgcagtc

SEQ ID NO: 14 - DNA, 21 bp, synthetic construct
  source 1..21: mol_type = other DNA
catgaccagttccacactctt

SEQ ID NO: 15 - DNA, 20 bp, synthetic construct
  source 1..20: mol_type = other DNA
ccttccttctgttgcttcca

SEQ ID NO: 16 - DNA, 19 bp, synthetic construct
  source 1..19: mol_type = other DNA
ttccaagagccacctccta

SEQ ID NO: 17 - DNA, 20 bp, synthetic construct
  source 1..20: mol_type = other DNA
gggttggaatgcaatttcgg

SEQ ID NO: 18 - DNA, 20 bp, synthetic construct
  source 1..20: mol_type = other DNA
tggtgtttacagttgggctg

SEQ ID NO: 19 - DNA, 21 bp, synthetic construct
  source 1..21: mol_type = other DNA
gttaagcagtacagccccaaa

SEQ ID NO: 20 - DNA, 23 bp, synthetic construct
  source 1..23: mol_type = other DNA
agggcatatccaacaacaaactt

SEQ ID NO: 21 - DNA, 20 bp, synthetic construct
  source 1..20: mol_type = other DNA
ttgtgccaagtctggagatg

SEQ ID NO: 22 - DNA, 22 bp, synthetic construct
  source 1..22: mol_type = other DNA
ctcagagcggatgaaggtaaag

SEQ ID NO: 23 - DNA, 20 bp, synthetic construct
  source 1..20: mol_type = other DNA
tgcctccctgagtacataca

SEQ ID NO: 24 - DNA, 21 bp, synthetic construct
  source 1..21: mol_type = other DNA
ctggtcttgctcttggtcttt

SEQ ID NO: 25 - DNA, 25 bp, synthetic construct
  source 1..25: mol_type = other DNA
gatatggactctgacccaagataag

SEQ ID NO: 26 - DNA, 21 bp, synthetic construct
  source 1..21: mol_type = other DNA
agcaatcctgtagttgatggg

SEQ ID NO: 27 - DNA, 23 bp, synthetic construct
  source 1..23: mol_type = other DNA
cagatgctacgaggttccatatc

SEQ ID NO: 28 - DNA, 21 bp, synthetic construct
  source 1..21: mol_type = other DNA
gtgtttcggacaacagcattc

SEQ ID NO: 29 - DNA, 22 bp, synthetic construct
  source 1..22: mol_type = other DNA
ggctataagggttcctcctttc

SEQ ID NO: 30 - DNA, 21 bp, synthetic construct
  source 1..21: mol_type = other DNA
tttctctccgtagatggacct

SEQ ID NO: 31 - DNA, 27 bp, synthetic construct
  source 1..27: mol_type = other DNA
agaacatcagaactacgcctaaacatg

SEQ ID NO: 32 - DNA, 21 bp, synthetic construct
  source 1..21: mol_type = other DNA
ccattcccctgtcgaatgtct

SEQ ID NO: 33 - DNA, 23 bp, synthetic construct
  source 1..23: mol_type = other DNA
ctgctgctatacactcaccaata

SEQ ID NO: 34 - DNA, 23 bp, synthetic construct
  source 1..23: mol_type = other DNA
tcagcacagatctccttgtttag

SEQ ID NO: 35 - DNA, 19 bp, synthetic construct
  source 1..19: mol_type = other DNA
gcctctgcctccccatatg

SEQ ID NO: 36 - DNA, 19 bp, synthetic construct
  source 1..19: mol_type = other DNA
ggccggaaatgtaggcaaa

SEQ ID NO: 37 - DNA, 22 bp, synthetic construct
  source 1..22: mol_type = other DNA
gctgacctggaagagaacatta

SEQ ID NO: 38 - DNA, 22 bp, synthetic construct
  source 1..22: mol_type = other DNA
ggtacaaccagaaagctcaaac

SEQ ID NO: 39 - DNA, 21 bp, synthetic construct
  source 1..21: mol_type = other DNA
ggtccactaccaacgaaatca

SEQ ID NO: 40 - DNA, 21 bp, synthetic construct
  source 1..21: mol_type = other DNA
ctcctttggatcagcacagaa

SEQ ID NO: 41 - DNA, 23 bp, synthetic construct
  source 1..23: mol_type = other DNA
tgaaatgattcctgcaagtccat

SEQ ID NO: 42 - DNA, 23 bp, synthetic construct
  source 1..23: mol_type = other DNA
tctccccactctttttcattgtg

SEQ ID NO: 43 - DNA, 21 bp, synthetic construct
  source 1..21: mol_type = other DNA
ctacgtcaggtttcagctacg

SEQ ID NO: 44 - DNA, 20 bp, synthetic construct
  source 1..20: mol_type = other DNA
tcagcagagccacattcttt

SEQ ID NO: 45 - DNA, 20 bp, synthetic construct
  source 1..20: mol_type = other DNA
gcaaagactggttcctgaga

SEQ ID NO: 46 - DNA, 22 bp, synthetic construct
  source 1..22: mol_type = other DNA
cttcacattggccttgaaatga

SEQ ID NO: 47 - DNA, 20 bp, synthetic construct
  source 1..20: mol_type = other DNA
gtgtacagcagcctgaagaa

SEQ ID NO: 48 - DNA, 22 bp, synthetic construct
  source 1..22: mol_type = other DNA
ctcgaacatccagcgtaagtaa

SEQ ID NO: 49 - DNA, 24 bp, synthetic construct
  source 1..24: mol_type = other DNA
catgtttgtgatgggcgtgaacca

SEQ ID NO: 50 - DNA, 24 bp, synthetic construct
  source 1..24: mol_type = other DNA
tttggctagaggagccaagcagtt

SEQ ID NO: 51 - DNA, 23 bp, synthetic construct
  source 1..23: mol_type = other DNA
aatgtcttgattgttgaggatat

SEQ ID NO: 52 - DNA, 20 bp, synthetic construct
  source 1..20: mol_type = other DNA
acaaagtcaggtttatagcc

SEQ ID NO: 53 - DNA, 22 bp, synthetic construct
  source 1..22: mol_type = other DNA
ctgtgtcaagtctggagatgag

SEQ ID NO: 54 - DNA, 24 bp, synthetic construct
  source 1..24: mol_type = other DNA
gcttgtcttgatccttgttcttac

SEQ ID NO: 55 - DNA, 18 bp, synthetic construct
  source 1..18: mol_type = other DNA
caaagccctcctggacaa

SEQ ID NO: 56 - DNA, 20 bp, synthetic construct
  source 1..20: mol_type = other DNA
tcacagtcctgccagtttag

SEQ ID NO: 57 - DNA, 19 bp, synthetic construct
  source 1..19: mol_type = other DNA
tgggtccatccatcgcata

SEQ ID NO: 58 - DNA, 21 bp, synthetic construct
  source 1..21: mol_type = other DNA
gaagtaggtgcgcccataacc